(19) United States
(12) Patent Application Publication
(10) Pub. No.: US 2025/0176907 A1
(43) Pub. Date: Jun. 5, 2025

(54) ELECTRONIC RING INCLUDING SENSORS FOR MONITORING HEALTH AND FITNESS PARAMETERS

(71) Applicant: ULTRAHUMAN HEALTHCARE PVT LTD, Bangalore South (IN)

(72) Inventors: Apoorv SHANKAR, Bangalore (IN); Yogansh NAMDEO, Bengaluru (IN); Anoop JAYAN, Bangalore (IN)

(73) Assignee: ULTRAHUMAN HEALTHCARE PVT LTD, Bangalore South (IN)

(21) Appl. No.: 19/045,830

(22) Filed: Feb. 5, 2025

Related U.S. Application Data
(63) Continuation of application No. 18/843,536, filed on Sep. 3, 2024, now abandoned.

(30) Foreign Application Priority Data
Mar. 4, 2022 (IN) 202241011816

(51) Int. Cl. A61B 5/00; A61B 5/0205; A61B 5/022

(52) U.S. Cl. CPC A61B 5/6802; A61B 5/02055; A61B 5/02208; A61B 5/4812; A61B 5/4815; A61B 2560/0238; A61B 2560/0252; A61B 2562/18; A61B 2562/182

(57) ABSTRACT

The proposed invention relates to an electronic ring. The electronic ring comprises an outer layer, a middle layer, and an inner layer. The middle layer is a Printed Circuit Board (PCB) comprising one or more sensors connected with a microcontroller and a battery. The sensors include a Photo Plethysmography sensor, a temperature sensor, and a motion sensor, to capture values of plurality of health and fitness parameters of a user. The PCB is fixed into position in the outer layer using an adhesive tape. The adhesive tape is thermally conducting and electrically insulating. Values of the health and fitness parameters are processed to determine secondary parameters that are further processed to generate a sleep score and a recovery score of the user.

### FIELD OF INVENTION

This application is a continuation and claims priority to U.S. application Ser. No. 18/843,536, filed Sep. 3, 2024, which claims priority to International Application No. PCT/IN2023/050185, filed Feb. 28, 2023, which claims priority to Indian Application No. 202241011816, filed Mar. 4, 2022, each of which is hereby incorporated herein by reference in its entirety.

### BACKGROUND

Regular health and fitness monitoring is important for accomplishing long term health and wellness goals. Traditionally, fitness monitoring required manual recording of one's physical exercise activities. However, such manual recording is inconvenient and often inaccurate.

Generally, detection of one's health parameters require stationary medical equipment such as an ECG machine, BP monitoring machine etc. Consequently, a person always on the move is unable to keep a track of his physical health parameters to maintain his health in a good condition, thereby disabling him to have more control on his daily lifestyle, physical activities and habits. Needless to say, lack of knowledge about one's own body and inability to make conscious data driven lifestyle changes impacts one's health and fitness in the long run.

With advancement in technology, portable machines for detection of body parameters have been developed. Such machines may utilise sensors for measurement of health and fitness parameters. A sensor converts a physical parameter into a signal that is read by an observer or by an instrument. A size of a sensor may range from nano-sensors implanted in the body, to magnetic resonance imaging scanners that occupy a large room. However, even the portable machines may be heavy, bulky and an inconvenient means for measurement of bodily parameters for extended periods.

For a user conscious of maintaining good health, it is desired that his key bodily parameters, indicative of his fitness, are measured on the move with more accuracy, precision and range while also being non-invasive. Thus, there remains a need for a wearable device for tracking and analysing health and fitness through trends of various health and metabolic parameters, thereby enabling users optimise their food intake, workout routines, sleep and other general lifestyle activities.

### OBJECTS OF THE INVENTION

A general objective of the present invention is to offer an efficient and reliable system for measurement of health and fitness parameters.

Another objective of the invention is to provide a cost-effective system for measurement of health and fitness parameters.

Yet another objective of the invention is to provide a non-invasive system for measurement of health and fitness parameters.

### SUMMARY OF THE INVENTION

The summary is provided to introduce aspects related to an electronic ring for monitoring health and fitness parameters of users. The electronic ring may be worn over a finger. The electronic ring may include different sensors for monitoring the health and fitness parameters of users.

The present invention relates to an electronic ring for monitoring fitness and health parameters of a user. The electronic ring may comprise an outer layer, a middle layer including a Printed Circuit Board (PCB) housing one or more sensors for capturing fitness and health parameters of the user, and an inner layer. The middle layer may be fixed with the outer layer using an adhesive tape. The adhesive tape may have thermally conducting and electrically insulating properties.

In one aspect, the adhesive tape may prevent leakage of current from a battery powering the one or more sensors and undesired transfer of current from external sources to the one or more sensors.

In one aspect, the adhesive tape may enable capturing of ambient temperature by an infrared based temperature sensor through the outer layer. The outer layer may be made of a thermally conductive material.

In one aspect, a microcontroller may be mounted on the PCB. The microcontroller may process values of the fitness and health parameters received from the one or more sensors to determine one or more secondary parameters including blood glucose value, heart rate variability, and sleep quality.

In one aspect, the one or more sensors may capture the fitness and health parameters on determining a sudden change in body temperature.

In one aspect, the one or more sensors may capture the fitness and health parameters on determining a variation in Photoplethysmography (PPG) readings of the user for an extended time period.

In one aspect, the microcontroller may process the values of the fitness and health parameters of the user to determine stages of sleep and a sleep score for the user.

In one aspect, the microcontroller may determine a recovery score for the user based on the stages of sleep.

In one aspect, the electronic ring may further comprise an accelerometer for picking periodic pulses of blood vessels and a microphone for capturing Korotkoff sounds, from a finger of the user, for determining Blood Pressure of the user.

Other aspects and advantages of the invention will become apparent from the following description, taken in conjunction with the accompanying drawings, illustrating by way of example the principles of the invention.

### BRIEF DESCRIPTION OF THE DRAWINGS

The accompanying drawings constitute a part of the description and are used to provide further understanding of the present invention. Such accompanying drawings illustrate the embodiments of the present invention which are used to describe the principles of the present invention. The embodiments are illustrated by way of example and not by way of limitation in the figures of the accompanying drawings in which like references indicate similar elements. It should be noted that references to “an” or “one” embodiment in this invention are not necessarily to the same embodiment, and they mean at least one. In the drawings:

FIG. 1 illustrates a front perspective view of an electronic ring, in accordance with an embodiment of the present invention;

FIG. 2 illustrates an exploded view of the electronic ring, in accordance with an embodiment of the present invention;

FIG. 3 illustrates a side view of a middle layer including a PCB used in the electronic ring, in accordance with an embodiment of the present invention; and

FIGS. 4(a) and 4(b) illustrate top view and side view respectively of the electronic ring placed on a wireless charger, in accordance with an embodiment of the present invention.

### DETAILED DESCRIPTION OF THE INVENTION

The detailed description set forth below in connection with the appended drawings is intended as a description of various embodiments of the present invention and is not intended to represent the only embodiments in which the present invention may be practiced. Each embodiment described in this disclosure is provided merely as an example or illustration of the present invention, and should not necessarily be construed as preferred or advantageous over other embodiments. The detailed description includes specific details for the purpose of providing a thorough understanding of the present invention. However, it will be apparent to those skilled in the art that the present invention may be practiced without these specific details.

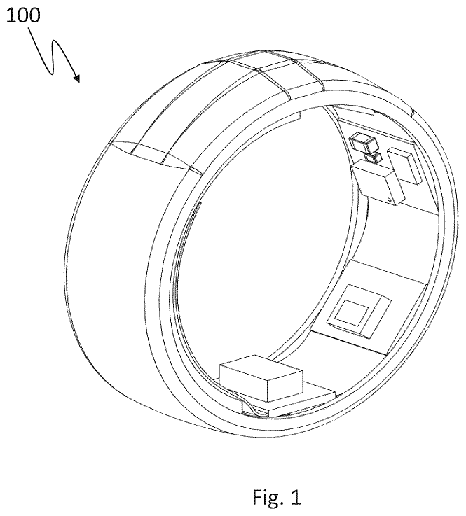

The proposed invention relates an electronic ring (100) for monitoring health and fitness parameters of users. The electronic ring (100) may be worn over a finger. FIG. 1 illustrates a front perspective view of the electronic ring (100), in accordance with an embodiment of the present invention. The electronic ring (100) may be developed in different sizes to fit over different fingers of different users. The electronic ring (100) may include different sensors for monitoring the health and fitness parameters of users. Types, placement, and working of the sensors used in the electronic ring (100) is described henceforth.

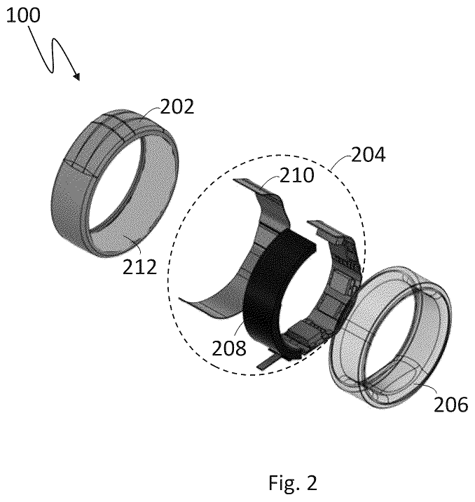

FIG. 2 illustrates an exploded view of the electronic ring (100), in accordance with an embodiment of the present invention. The electronic ring (100) may comprise an outer layer (202), a middle layer (204), and an inner layer (206). The outer layer (202) may be made of a rigid, antirust or thermally conductive material, or a material having all such properties. The middle layer (204) may be positioned between the outer layer (202) and the inner layer (206). The middle layer (204) may include a Printed Circuit Board (PCB) (208) and a battery (210). The PCB (208) may be flexible, semi-flexible, or rigid. The PCB (208) may house one or more sensors to capture a plurality of health and fitness parameters of a user.

The inner layer (206) of the electronic ring (100) positioned below the PCB (208) may come in contact of the user's finger once the user wears the electronic ring (100). The inner layer (206) may be made of a semi-transparent, translucent, or completely transparent, water-resistant material, such as glass, plastic, epoxy resin, or silicone. The inner layer (206) may be transparent to a wide range of wavelengths in the electromagnetic spectrum.

In one implementation, an adhesive tape (212) may be disposed on an inner side of the outer layer (202) between the middle layer (204) and the outer layer (202). The adhesive tape (212) may be used to fix the PCB (208) in the outer layer (202) such that the one or more sensors are aligned in their appropriate positions for capturing most accurate readings. The adhesive tape (212) may have thermally conducting and/or electrically insulating properties. Thermally conducting property of the adhesive tape (212) may enable transmission of readings of ambient temperature to an infrared based temperature sensor housed in the PCB (208).

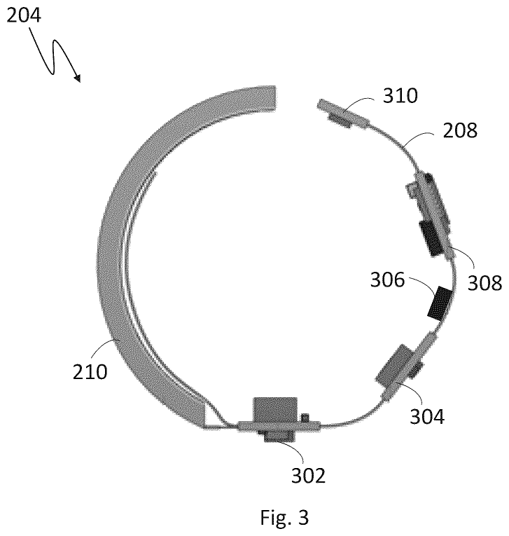

FIG. 3 illustrates a side view of the middle layer (204) including the PCB (208), in accordance with an embodiment of the present invention. The PCB (208) may include a Photoplethysmography (PPG) sensor (302). The PPG sensor (302) may calculate blood oxygen saturation (SPO2) level and heart rate of a user. The heart rate of the user may be measured by detecting volumetric variations of blood circulation of the user. The PPG sensor (302) focusses lights of different wavelength on the user's skin and simultaneously measures the reflected light signals through a photodetector. The transmission and capturing of reflection of the light by the PPG sensor (302) is done through the inner layer (206). The inner layer (206) may be made transparent to allow the PPG sensor (302) to obtain reading from the finger of the user.

The PCB (208) may comprise a temperature sensor (304) to measure body temperature of a user. The temperature sensor (304) may be an infrared based temperature sensor. The temperature sensor (304) may measure infrared energy emitted by the user. The infrared energy emitted by the user may be focussed onto one or more photodetectors present in the temperature sensor (304), whereby the infrared energy is converted into an electrical signal to detect body temperature of the user. The temperature sensor (304) may be positioned on the PCB (208) such that one side of the temperature sensor (304) faces surroundings of the user to detect ambient temperature through the outer layer (202) and another side of the temperature sensor (304) faces the user's skin to detect body temperature of the user through the inner layer (206). The inner layer (206) may be made of a thermally non-conductive and a Far-Infrared (FIR) transparent material to isolate one side of the temperature sensor (304) while the adhesive tape (212) may provide thermal interface with the outer layer (202) for measurement of ambient temperature. In an implementation, the adhesive tape may be made of a polyester based backing material. Value of the ambient temperature may be transmitted through the outer layer (202) and the adhesive tape (212), to the temperature sensor (304). The readings of the ambient temperature may be used to calibrate readings of the temperature sensor (304) for accurate sensing of body temperature of the user.

In one embodiment, presence of a thermally conductive outer layer (202) in the electronic ring (100) helps in detection of ambient temperature by the temperature sensor (304) without affecting aesthetics and durability of the outer layer (202) of the electronic ring (100). The outer layer (202) may be separated from the user's skin through the middle layer (204) and the inner layer (206), and not be in direct contact with the user's skin. In another embodiment, the adhesive tape (212) may only be positioned in contact with the outer layer (202) closer to where the temperature sensor (304) is positioned in the middle layer (204) such that the user's finger skin remains completely thermally disconnected with this portion of the outer layer (202). Thermally disconnecting the user's finger from the temperature sensor (304) helps in capturing a more accurate reading of the ambient temperature without unwanted noise.

In another embodiment, the adhesive tape (212) may have electrically insulating property and may prevent leakage of current from the battery (210) powering the one or more sensors, through the outer layer (202). Further, the adhesive tape (212) may prevent undesired transfer of current from external sources to the one or more sensors. For example, the adhesive tape (212) would prevent transfer of static charge coupling with the outer layer (202), to an assembly of the PCB (208) and prevent damage to sensitive components integrated in the PCB (208).

The PCB (208) may comprise a motion sensor (306) to detect gestures and day to day movement of the user. The motion sensor (306) may be one or more of an acceleration sensor, gyroscope, and magnetometer.

A microcontroller (308) may be mounted on the PCB (208). The one or more sensors mounted on the PCB (208) may be connected to the microcontroller (308). The one or more sensors may transmit values of the plurality of health and fitness parameters to the microcontroller (308), in real-time. The microcontroller (308) may obtain values of the plurality of health and fitness parameters from the one or more sensors based on internal and external triggers associated with the one or more sensors.

The internal triggers may include time-based triggers generated at a pre-determined frequency for data collection. The external triggers may include motion-based triggers, temperature-based triggers, and heart rate-based triggers. The motion-based triggers may include actions by the user such as specific gestures, a specific pattern of taps on the electronic ring (100), sudden jerks experienced by the user, and other actions such as walking, running, and swimming. The temperature based triggers may include sudden spike or drop in body temperature of the user. The heart based triggers may include rapid variation in readings captured by the PPG sensor (302) over an extended periods of time. The microcontroller (308) may also store values of the plurality of the health and fitness parameters in its own memory or a separate memory element mounted on the PCB (208).

The microcontroller (308) may analyse and process values of the plurality of health and fitness parameters received from the one or more sensors to determine secondary parameters of the user. The secondary parameters may include blood glucose value, heart rate variability and sleep quality. The heart rate variability may be calculated as a variation in the time interval (measured in milliseconds) between consecutive heartbeats of the user. The sleep quality may include details of different sleep states of the user such as REM (Rapid Eye Movement) sleep, light sleep, and deep sleep. The secondary parameters may further be processed to generate a sleep score and recovery/readiness score for the user. The sleep score may indicate quality of sleep during a duration of sleep session of the user. The recovery/readiness score may indicate if the body of the user has rested well the previous day or the body requires more rest for a particular activity to be done on the day. Additionally, a blood glucose value of the user may be determined based on the detection of the heart rate of the user.

In another embodiment, the electronic ring (100) may be used to accurately predict whether the user is having fever or not. The microcontroller (308) may compare the ambient temperature with the body temperature. When reading of the body temperature is higher than normal body temperature i.e. 98.6 degree Fahrenheit, but the reading of the ambient temperature is also high, the electronic ring (100) may still report that the user body temperature to be normal by factoring in the reading of the ambient temperature. The electronic ring (100) may also be utilised to predict any disease accurately.

In one implementation, the microcontroller (308) may obtain readings of heart rate, body temperature, and motion from the one or more sensors, at a pre-determined frequency. Readings captured from the one or more sensors may be cleaned for undesirable or incorrect values before processing. Current readings and readings from the one or more sensors over a period of time, such as for a duration of 4 to 5 hours, may be analysed for detection of the sleep pattern over the period of time. The sleep pattern may further be processed using empirically determined constants and readings captured from the one or more sensors may be weighted, to determine stages of sleep. Stages of sleep may be classified into an array of values indicating an awake state, intermediate state, and light sleep state of the user. An array of values may be generated based on classifier values. Classifier data-array may be re-classified based on the pattern of awake state and light sleep state to determine if false positives are recorded for any of the classified data. The classifier data-array may be presented to the user with correctly classified Awake, Light, REM, and deep sleep times and actual awake times in between sleep stages. The entire classified data may then be clubbed into a total sleep time. The sleep time may be a time between what is classified as sleep start time and sleep end time excluding the awake times in between.

In one implementation, a sleep hypnogram may be generated based on the array of classified values. The array of classified values within the sleep window may be chunked into sleep stages to generate the sleep hypnogram. The classified values may be chunked into four stages. The four stages include awake, light sleep, Rapid Eye Movement (REM) sleep, and deep sleep. The sleep stages may be determined by chunking the array of classified values based on the arrangement of the type of the classifier data in the data-array and on specific arrangements of the classifier data.

Similarly, other health and fitness parameters such as Blood Pressure (BP), and stress levels of the user may be measured by including other sensors on the PCB (208). A haptic piezo electric sensor may determine BP by gathering Korotkoff sounds from arteries and measuring pressure near the skin.

In one implementation, the electronic ring (100) may be fixed snugly to the finger of the user to imitate the effect caused by the inflated cuff of a sphygmomanometer. The PCB (208) may comprise a sensitive accelerometer trained to pick up periodic pulses of the blood vessel in the finger of the user. The PCB (208) may further comprise a sensitive microphone. The accelerometer may be used in conjunction with the microphone trained to listen to 5 phases of the Korotkoff sounds. The measurement of the Korotkoff sounds may be processed by the microcontroller (308) to determine blood pressure of the user.

A hydration and bio-potential sensor may detect stress levels of the user by measuring sweat levels and changes in skin conductance of the user.

The PCB (208) may be connected with an external user device through a wireless module (310). The wireless module (310) may work on one or more of Bluetooth and Near Field Communication (NFC). The wireless module (310) may be mounted on the PCB (208) to wirelessly communicate the plurality of health and fitness parameters and the secondary parameters to the external user device, such as a smartphone or a laptop. The external user device may act as a notification means for the user to access readings of the plurality of health and fitness parameters and the secondary parameters in a visual or audible format. In another implementation, the PCB (208) may be configured to connect with the external user device through a cloud based platform via a network(s).

The battery (210) may be used to power the one or more sensors, the micro-controller (308), and the wireless module (310) in the electronic ring (100).

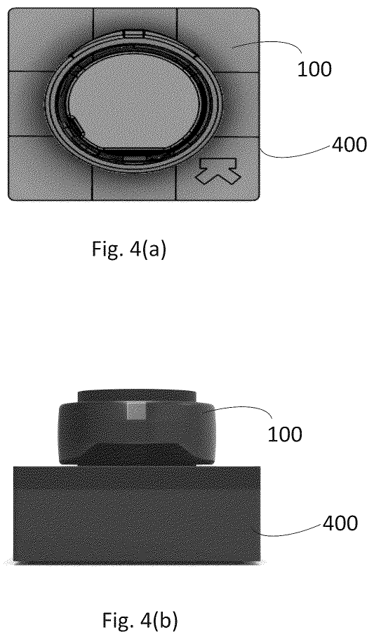

FIGS. 4(a) and 4(b) illustrate top view and side view respectively of the electronic ring (100) placed on a wireless charger (400), in accordance with an embodiment of the present invention. The electronic ring (100) may be wirelessly chargeable using the wireless charger (400). For wireless charging, the electronic ring (100) may comprise a wireless charging coil. The wireless charging coil may be positioned above or below the PCB (208). Electromagnetic field generated by a coil present in the wireless charger (400) may get coupled with the wireless charging coil of the electronic ring (100) when the electronic ring (100) is present above the wireless charger (400). Through coupling of the electromagnetic field, power may be received and stored in the battery (210). The wireless charger (400) may itself include a battery of capacity sufficient to charge the battery (210) of the electronic ring (100) a few times.

The electronic ring (100) may be worn by a user at all times so that his health and fitness parameters are continuously tracked and reported to him. The electronic ring (100) provides a cost effective mean to non-invasively measure health and fitness parameters of a user in real time. With the data obtained from the electronic ring (100), a user may be able to track changes in his lifestyle, activities, and habits.

In the above detailed description, reference is made to the accompanying drawings that form a part thereof, and illustrate the best mode presently contemplated for carrying out the invention. However, such description should not be considered as any limitation of scope of the present unit. The structure thus conceived in the present description is susceptible of numerous modifications and variations, all the details may furthermore be replaced with elements having technical equivalence.

1. An electronic ring for monitoring fitness and health parameters of a user, the electronic ring comprising:
an outer layer, a middle layer including a Printed Circuit Board (PCB) housing one or more sensors for capturing fitness and health parameters of the user, and an inner layer,
wherein the middle layer comprises a plurality of light and motion based sensors, battery and wireless charging coil,
and the middle layer is fixed with the outer layer using an adhesive tape, the adhesive tape has thermally conducting and electrically insulating properties.

2. The electronic ring as claimed in claim 1, wherein the adhesive tape prevents leakage of current from a battery powering the one or more sensors and undesired transfer of current from external sources to the one or more sensors.

3. The electronic ring as claimed in claim 1, wherein the adhesive tape enables capturing of ambient temperature by an infrared based temperature sensor, through the outer layer, and wherein the outer layer is made of a thermally conductive material.

4. The electronic ring as claimed in claim 1, comprising a microcontroller mounted on the PCB, the microcontroller processes values of the fitness and health parameters received from the one or more sensors to determine one or more secondary parameters including blood glucose value, heart rate variability, and sleep quality.

5. The electronic ring as claimed in claim 1, wherein the one or more sensors capture the fitness and health parameters on determining a sudden change in body temperature.

6. The electronic ring as claimed in claim 1, wherein the one or more sensors capture the fitness and health parameters on determining a variation in Photoplethysmography (PPG) readings of the user for an extended time period.

7. The electronic ring as claimed in claim 4, wherein the microcontroller processes the values of the fitness and health parameters of the user to determine stages of sleep and a sleep score for the user.

8. The electronic ring as claimed in claim 7, wherein the microcontroller determines a recovery score for the user based on the stages of sleep.

9. The electronic ring as claimed in claim 1, comprising an accelerometer for picking periodic pulses of blood vessels and a microphone for capturing Korotkoff sounds, from a finger of the user, for determining Blood Pressure of the user.

10. The electronic ring as claimed in claim 1, wherein the temperature sensor measures infrared energy emitted by the user and focuses it onto one or more photodetectors for conversion into an electrical signal to detect body temperature.

11. The electronic ring as claimed in claim 1, wherein the adhesive tape is made of a polyester-based backing material to provide thermal interface with the outer layer.

12. The electronic ring as claimed in claim 4, wherein the microcontroller analyses motion-based, temperature-based, and heart rate-based triggers for data collection.

13. The electronic ring as claimed in claim 1, wherein the temperature sensor is calibrated using ambient temperature readings to ensure accurate sensing of the body temperature of the user.

14. The electronic ring as claimed in claim 1, wherein the outer layer is separated from the user's skin to prevent thermal interference during ambient temperature sensing.

15. The electronic ring as claimed in claim 7, wherein a sleep hypnogram is generated based on classified values of sleep stages within a predefined sleep window.

16. The electronic ring as claimed in claim 1, wherein the PCB comprises a motion sensor configured to detect user gestures and daily movements.

17. The electronic ring as claimed in claim 1, wherein the adhesive tape prevents transfer of static charge coupling with the outer layer, to an assembly of the PCB and prevent damage to sensitive components integrated in the PCB.

18. The electronic ring as claimed in claim 1, wherein the motion sensor comprises one or more of an acceleration sensor, gyroscope, and magnetometer to detect to detect gestures and day to day.

19. The electronic ring as claimed in claim 1, wherein the microcontroller cleans raw data from the sensors to remove incorrect or undesirable values before processing.

20. The electronic ring as claimed in claim 1, wherein the PCB is configured to connect to an external user device through a cloud-based platform for real-time monitoring and data analysis.
